(19) United States
(12) Patent Application Publication
(10) Pub. No.: US 2022/0154120 A1
(43) Pub. Date: May 19, 2022

(54) Hanging Cell Culture Millifluidic Device

(71) Applicant: Northeastern University, Boston, MA (US)

(72) Inventors: Eno Essien Ebong, Milton, MA (US); Ian Harding, Boston, MA (US); Ira M. Herman, Boston, MA (US); Abigail N. Koppes, Charlestown, MA (US); Alex Caraballo, Silver Spring, MD (US); Nicholas O'Hare, Boston, MA (US); Mark Vigliotti, Levittown, PA (US)

(21) Appl. No.: 17/454,768

(22) Filed: Nov. 12, 2021

Related U.S. Application Data
(60) Provisional application No. 63/114,439, filed on Nov. 16, 2020.

(51) Int. Cl. C12M 3/00; C12M 3/06; C12M 1/00; G01N 33/50

(52) U.S. Cl. CPC C12M 23/48; C12M 23/16; G01N 33/5088; C12M 23/22; C12M 23/38; C12M 23/44

(57) ABSTRACT

Devices and systems for cell culture analysis are provided. A device for cell culture analysis includes a first component comprising a receptacle configured to receive a cell culture insert having an apical surface and a basal surface and a second component. The device further includes an inlet port disposed at at least one of the first and second components and an outlet port disposed at at least one of the first and second components. The first component and second component are releasably couplable and configured to define a flow path from the inlet port to the outlet port when in a coupled state. The flow path is at least partially defined by a surface of the second component, and the first component is configured to expose the basal surface of the cell culture insert to the flow path.

### RELATED APPLICATION(S)

This application claims the benefit of U.S. Provisional Application No. 63/114,439, filed on Nov. 16, 2020. The entire teachings of the above application(s) are incorporated herein by reference.

### GOVERNMENT SUPPORT

This invention was made with government support under Grant No. HL125499 from the National Institutes of Health. The government has certain rights in the invention.

### BACKGROUND

Neurological disorders are the second leading cause of death worldwide and the leading cause of daily-adjusted life years, a sum of the years of potential life lost and the years of productive life lost. Over the past several decades, numerous studies have identified correlations between many neurological disorders, such as Alzheimer's, stroke, multiple sclerosis, traumatic brain injury, and dysfunction of the blood-brain barrier (BBB), a complex, multicellular structure composed of endothelial cells (ECs), pericytes (PCs), astrocytes (ACs), neurons, and microglia. These studies, among others, suggest that BBB dysfunction in neurological disorders may even contribute to their pathology. Therefore, identifying the regulatory mechanisms of BBB integrity may provide therapeutic targets for neurological disorders.

Proper function of the BBB requires constant communication between brain ECs and supportive cells such as PCs, ACs, neurons, and microglia. In particular, PCs and ACs have been shown to regulate the expression of endothelial transporters and tight junction proteins, thereby promoting reduced permeability within the BBB. For example, the addition of PCs and ACs to brain EC monolayers has been shown to increase the expression of the tight junction proteins zona occludins-1 (ZO-1), claudin-5, and occludin. Supportive PCs and ACs have also been shown to impact EC caveolin-1, implicated in endocytosis, and the adherens junction protein VE-cadherin, although this has been less studied. While many studies have identified beneficial roles of ACs and PCs in BBB function, others have found contradicting results, including in diseased state. Thus, the relationship between ECs and neighboring PCs and ACs is not clear.

Brain EC exposure to shear stress has also been shown to improve barrier integrity. For instance, it was found that the application of shear stress at a rate of 14 dynes/cm² reduces permeability and increases the expression of ZO-1 and claudin-5. However, this study utilized bovine brain ECs, limiting its physiological relevance. Other studies have similarly investigated the relationship between shear stress and function of brain ECs and BBB, but typically use non-human and/or immortalized ECs, apply sub-physiological shear stress magnitudes, or fail to include relevant supportive cells including PCs and ACs. Interestingly, it has also been shown that brain ECs may not respond to shear stress application in the similar manner to other vascular beds, particularly due to their resistance to elongation and alignment in the direction of shear stress, a classical endothelial response to fluid flow.

There exists a need for methods and devices that can provide for improved BBB fluidic models to enable further research.

### SUMMARY

Cell culture devices and systems that can provide for improved modeling and analysis of complex cell cultures, such as a BBB fluidic model, are provided.

A device for cell culture analysis includes a first component comprising a receptacle and a second component. The receptacle is configured to receive a cell culture insert having an apical surface and a basal surface (e.g., a hanging cell culture insert). The device further includes an inlet port and an outlet port, each of which is disposed at at least one of the first and second components. The first component and second component are releasably couplable and configured to define a flow path from the inlet port to the outlet port when in a coupled state. The flow path is at least partially defined by a surface of the second component, and the first component is configured to expose the basal surface of the cell culture insert to the flow path.

The device can further include a third component configured to engage with the first component to enclose the cell culture insert in the receptacle.

The surface of the second component and a complementary surface of the first component can define a channel that at least partially defines the flow path. At least one of the first and second components can include at least one channel support member configured to engage a complementary structure at the other of the first and second components to maintain a height of the channel when pressure is applied to the device. For example, at least two channel support members can be included in the device, each disposed at opposing ends of the channel. One of the at least two channel support members can be disposed adjacent to the inlet port and the other of the at least two channel support members can be disposed adjacent to the outlet port. The channel can be a millifluidic channel.

The receptacle can include an alignment structure configured to maintain a position of the basal surface of the cell culture insert with respect to the flow path. At least one of the first and second components can include at least one alignment member configured to engage a complementary structure at the other of the first and second components to align the first and second components for coupling. The at least one alignment member can be further configured to be a channel support member.

Any or all of the first, second, and third components can include or be formed from a transparent material to enable observation. Any or all of the first, second, and third components can be reusable, sterilizable, autoclavable, or a combination thereof. At least one sealing member can be disposed between the first and second components and configured to maintain a pressure of the flow channel. At least one sealing member can be disposed between the first and third components to enclose the cell culture insert.

The first component can include a plurality of receptacles, each receptacle configured to receive a cell culture insert. The receptacles can be arranged in a linear configuration with a single channel to provide a common flow path. Alternatively, the surface of the second component and a complementary surface of the first component can define at least two channels that at least partially define a flow path to provide for a parallel configuration.

A system for cell culture analysis includes a device and at least one pump in fluidic communication with the inlet port. The pump can be configured to supply a fluid flow to the fluid path at a flow rate that induces shear stress of cells disposed at the basal surface. For example, the flow rate can be of about 0.1 ml/min to about 120 ml/min.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is an expanded view of an example cell culture analysis device.

FIG. 2 is a top view of a first component of the cell culture analysis device of FIG. 1.

FIG. 3 is a bottom view of the first component shown in FIG. 2.

FIG. 4 is a top view of a second component of the cell culture analysis device of FIG. 1.

FIG. 5 is an expanded view of another example cell culture analysis device.

FIGS. 6A and 6B are illustrations of modelled fluid flow through the cell culture analysis device of FIG. 5 in a perspective view (FIG. 6A) and cross-section view (FIG. 6B).

FIGS. 7A and 7B are illustrations of modelled fluid flow through the cell culture analysis device of FIG. 1 in a vertical cross-section view (FIG. 7A) and a bottom-up view (FIG. 7B).

FIG. 8 is a schematic of a hanging cell culture insert.

FIG. 9 is a schematic of a cross-section of a cell culture analysis device and its inclusion in a cell culture analysis system.

FIGS. 10A and 10B are flow diagrams illustrating a process for using an example device for flow experiments (FIG. 10A) and a process for experiments performed in static conditions (FIG. 10B).

FIGS. 11A and 11B are photos of live tracking results of fluorescent microspheres in a static device (FIG. 11A) and in an example cell culture flow device (FIG. 11B), which validated the expected flow patterns predicted by the computational model shown in FIGS. 7A and 7B.

FIG. 12 is an expanded view of yet another example cell culture analysis device.

### DETAILED DESCRIPTION

A description of example embodiments follows.

Cell culture devices and systems that can provide for improved modeling and analysis of complex cell cultures, such as a blood-brain barrier (BBB) fluidic model, are described.

Early multicellular, in vitro BBB models were typically cultured on Transwell® inserts (Corning, Inc., Corning, N.Y.) and maintained in static conditions. These models thus had limited physiological relevance due to a lack of shear stress created by fluid flow. With advances in microfluidic research, static BBB models were adapted to microfluidic formats, most commonly via the use of polydimethylsiloxane (PDMS). However, despite the advances and successful applications of PDMS-based microfluidic BBB models, they are often complicated by issues of limited nutrient diffusion and air bubble formation and therefore require sufficient microfluidic expertise to utilize. There exists a need to develop a multicellular BBB model that retains the application of fluid flow and overcomes the challenges associated with common PDMS microfluidics.

Devices and systems are provided that can be used for modelling complex cell cultures while overcoming common limitations of previous models, such as issues with nutrient diffusion and air bubble formation. In particular, millifluidic devices that allow for the application of physiological levels of shear stress while maintaining ease of use and compatibility with downstream analytical molecular biology techniques are described. With such devices, the study of complex cell cultures can be achieved. Such devices are configured to receive cell culture inserts, thereby allowing for the establishment of a physiologically relevant model on the insert prior to use with the millifluidic device. For example, as further described in the Exemplification section herein, a BBB model was established that included primary human brain microvascular endothelial cells (HBMECs), human PCs, and human ACs. A prototype device enabled a study to clarify the impacts of PCs and ACs on BBB phenotype and also to identify the impact of flow on BBB integrity (see Example 1, herein). The provided devices are configured to accept the cell culture insert to enable further modeling and study, including the impact of PC and AC addition and shear stress exposure. As further described in the Exemplification section herein, sample devices were used to analyze the established BBB model via dextran permeability assays, cell alignment, and the expression of permeability-regulating proteins via western blotting and immunocytochemistry.

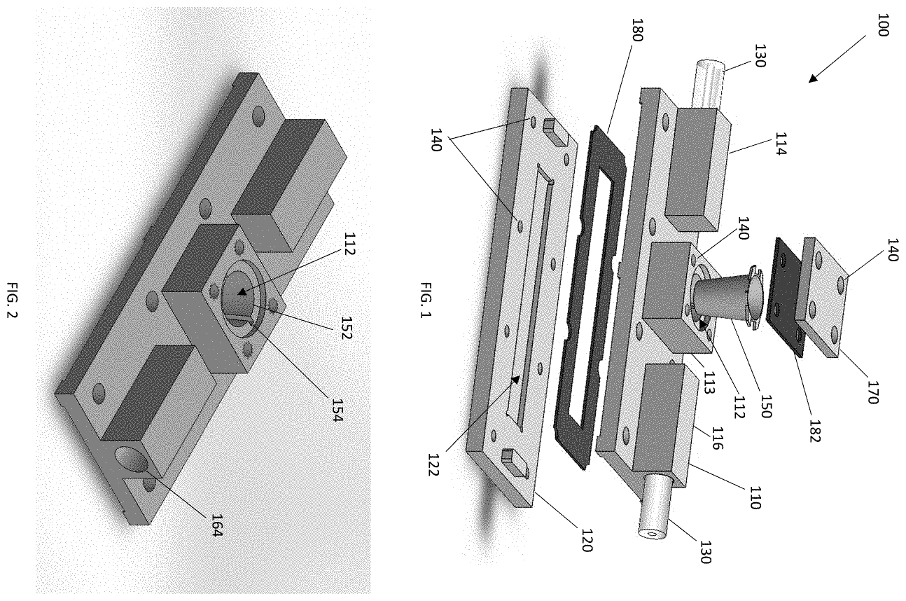

An example cell culture analysis device is shown in FIG. 1. The device 100 includes a first component 110 that includes a receptacle 112 configured to receive a cell culture insert 150. The receptacle 112 can be defined by a receptacle structure 113. As illustrated, the cell culture insert 150 is a hanging cell culture insert, such as a Transwell® Insert (Corning Inc., Corning, N.Y.).

A schematic of an example hanging cell culture insert is shown in FIG. 8. A hanging cell culture insert 350 typically includes a membrane 352 or other structure on which cells may be cultured and which has an apical surface 354 and a basal surface 356. The insert, when placed in a well 370, provides for an apical chamber 355 and a basal chamber 357. Cells 360 can be cultured at either or both of the apical and basal surfaces 354, 356.

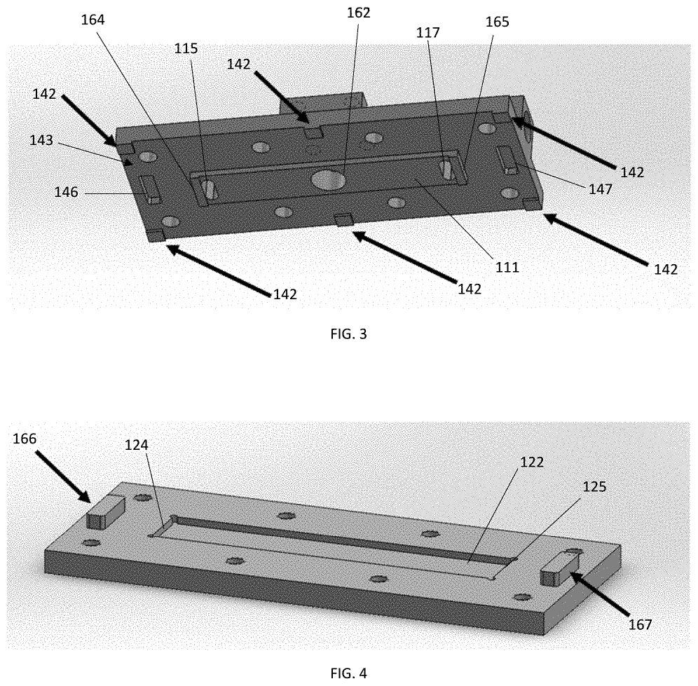

Returning to FIG. 1, the device 100 further includes a second component 120. At least one of the first and second components 110, 120 includes an inlet structure 114, and at least one of the first and second components 110, 120 includes an outlet structure 116. As illustrated in FIGS. 1-4, the inlet and outlet structures 114, 116 are defined by the first component 110; however, the structures can alternatively be defined by the second component 120. The inlet and outlet structures are configured to provide for a fluidic coupling, such as a connection with tubing 130, to supply fluid to the device and remove fluid from the device. The inlet structure 114 further defines an inlet port 115, and the outlet structure 116 further defines an outlet port 117 (FIG. 3).

The first and second components 110, 120 are releasably couplable and configured to define a flow path (as indicated by arrow A in FIG. 9) from the inlet port 115 to the outlet port 117 when in a coupled state. The flow path is at least partially defined by a surface 122 (FIG. 4). As also shown in FIG. 9, the first component 110 is configured to expose a basal surface 156 of the cell culture insert 150 to the flow path.

The device 100 can optionally include a third component 170 configured to engage with the first component 110 to enclose the cell culture insert 150 within the receptacle 112. The third component 170 can assist with retaining the cell culture insert in place, particularly as the insert is exposed to fluid flow. To provide for a fluid tight coupling and maintain pressure within the flow path, the device 100 can include one or more sealing members 180, 182, for example, gaskets, that can be disposed between the first, second, and third components.

As best seen in FIGS. 3 and 4, the first and second components 110, 120 can include complementary surfaces 111, 122 to define a channel 182 (FIG. 9) that defines, at least in part, the flow path A. For example, as illustrated, the first component 110 includes a projected surface 111 that is complementary to the recessed surface 122 of the second component 120. A channel 182 can be defined by the complementary surfaces 111, 122 to provide for a controlled fluid flow at the insert aperture 162 at which a basal surface of a cell culture insert resides. While the surface 111 of the first component 110 is shown as projected (or outdented) and the surface 122 of the second component 112 is shown as recessed (or indented) in FIGS. 3 and 4, alternative arrangements are possible. For example, the first component can include a recessed surface and the second component can include a complementary projected surface. The complementary surfaces can provide for a channel with a particularly defined height, width, and length to provide for controllable, consistent and repeatable flow conductions in the flow path.

Optionally, at least one of the first and second components includes at least one channel support member configured to engage a complementary structure at the other of the first and second components to maintain a height of the channel 182. As illustrated in FIGS. 3 and 4, channel support members 164, 165 are disposed at the first component 110, but may alternatively be disposed at the second component 112. The channel support members 164, 165 are configured to engage with the edges 124, 125 of the recessed portion defining the surface 122 of the second component. As illustrated in FIG. 4, the channel support members 164, 165 are disposed at opposing ends of the surface 111 and adjacent to the inlet 115 and outlet 117. Such a configuration can advantageously provide for structural support to the channel while not interfering with flow conditions between the inlet and outlet. The channel support members 164, 165 can further advantageously serve as alignment features during assembly of the device by being receivable within and abutting the defining edges of the surface 122.

Additional members that provide for alignment and/or channel support can be included in the device. For example, as illustrated in FIGS. 3 and 4, the second component 120 further includes structures 166, 167, which are configured to engage with complementary structures 146, 147 of the first component 110. The structures 146, 147, 166, 167 can serve as additional alignment features and provide for additional support to maintain a height of the channel upon assembly of the device. As illustrated in FIGS. 3 and 4, each of the first and second components includes a combination of projected and recessed features. For example, the first component 110 includes both projected features (e.g., structures 164, 165) and recessed features (e.g., structures 146, 147). Including a combination of projected and recessed features on each of the first and second components can provide for more robust support and easier alignment.

The device 100 can further include fastening structures 140. As shown in FIG. 1, fastening structures 140 are disposed at each of the first, second, and third components 110, 120, 170. The fastening structures can be, for example, bores configured to receive screws or pegs, joint fasteners (e.g., tenon and mortise structures), press-fit structures, or other structures capable of releasable coupling. To prevent against overtightening of fasteners, such as can occur when the components are coupled by threaded screws, additional supports 142 can be included, as illustrated in FIG. 3. As illustrated, supports 142 are projections disposed about an outer perimeter of a lower surface 143 of the first component 110. The supports can provide for additional device integrity when the first and second components are in an assembled state. Supports can also be included between the first and third components.

The channel can be a millifluidic channel. For example, the channel can have a width of about 1 mm to about 50 mm, or of about 5 mm to about 20 mm, or of about 10 mm to about 15 mm (e.g., 9.5 mm, 10 mm, 11 mm, 11.5 mm, 12 mm, 15.5 mm). The channel can have a height of about 0.5 mm to about 5 mm, or about 1 mm to about 3 mm, or of about 1.5 mm (e.g., 1.1 mm, 1.3 mm, 1.5 mm, 1.7 mm).

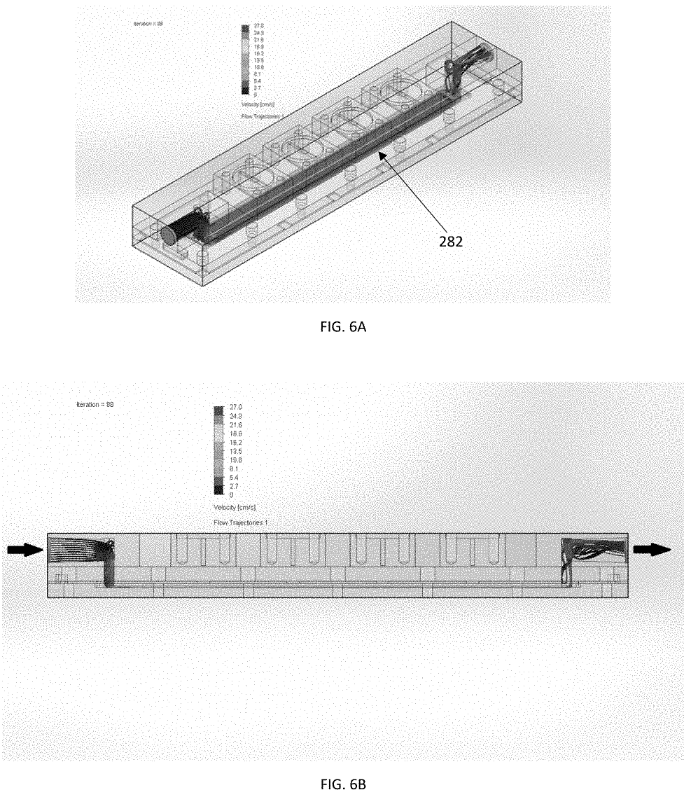

As illustrated, the surfaces 111, 122 providing for the channel 182 are of an elongated, substantially rectangular geometry. A substantially rectangular geometry can advantageously allow for flow conditions to stabilize in the channel prior to fluid flow reaching the aperture 162. When fluid first enters the channel, a velocity profile of the fluid flow can change suddenly (as illustrated, for example, in FIG. 6B). As fluid moves through the channel, a constant velocity profile can be achieved that can mimic a physiological environment (e.g., laminar flow at the basal surface of the cell culture insert). As illustrated, the aperture 162 is disposed substantially equidistant from the inlet 115 and outlet 117 and is substantially centered with respect to both a length and width of the channel. Such a configuration can provide for the basal surface of the cell culture insert to be exposed to a fluid flow that is undisturbed by boundary conditions within the channel. The aperture can alternatively be disposed off-center, and the channel can alternatively be of other geometries to provide for other flow conditions.

As further illustrated in FIG. 2, the receptacle 112 defined by the first component 110 can include one or more alignment features 152, 154. For example, the receptacle 112 includes the alignment feature of a lip 152 configured to complement an upper edge of a hanging cell culture insert and position the hanging cell culture insert within the receptacle such that a basal surface of the insert is flush with the surface 111 or substantially in a same plane with the inlet 115 and outlet 117. Such a configuration can advantageously provide for cells disposed on the insert to be exposed to laminar flow in the channel. As illustrated, the receptacle 112 further includes at least one alignment feature 154, such as one or more notches, configured to engage with a complementary structure of the hanging cell culture insert. The alignment feature 154 (e.g., notches) can allow for consistent and repeatable insertion of the inserts into the device, and a notch depth can allow for coincident alignment between the basal surface of the insert with the surface 111. The notches 154 can further prevent rotation or other movement of the insert within the receptacle.

The first component, second component, third component, or any combination thereof can include or be formed of a transparent material. Examples of suitable transparent materials include acrylic (which can be machined) and clear resin (which can be 3D printed). The transparent material can advantageously provide for ease of observation of cells disposed at the membrane of the insert during use of the device. The first, second, and/or third component can be formed of stainless steel. Components comprising stainless steel can advantageously provide for durability and sterility. Any of the first, second, and third components can be formed of a combination of stainless steel and transparent material. For example, portion(s) of any of the first, second, and third components can comprise a transparent material to provide for one or more optical windows while a remainder of the component comprises stainless steel.

The first component, second component, or both can be reusable, sterilizable, autoclavable, or a combination thereof. For example, acrylic components can be sterilized by application of alcohol, ultraviolet light, or a combination thereof, and stainless steel components can be autoclavable. The releasable two-piece configuration of the first and second components can provide for ease of cleaning of the flow channel for reuse of the device, in addition to ease of manufacturing.

As illustrated in FIGS. 1 and 9, the device can be configured for fluidic coupling with other components of a system. As illustrated in FIG. 1, the inlet and outlet structures include apertures (e.g., aperture 164 at outlet structure 116) configured to receive tubing 130. As illustrated in FIG. 9, such fluid connections can provide for connection to a fluid pump 400, optionally connected to a controller 410, and a receptacle 420. The pump can be configured to supply a fluid flow to the fluid path at a flow rate that induces shear stress of cells disposed at the basal surface. The flow rate can be of about 0.1 ml/min to about 120 ml/min. Examples of suitable pumps includes peristaltic pumps and centrifugal pumps.

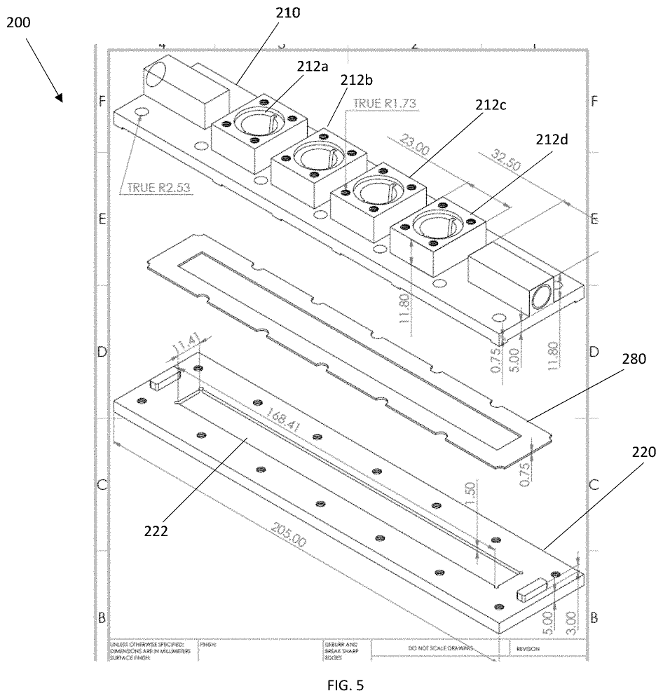

Another example of a cell culture analysis device is shown in FIG. 5. The device 200 accommodates a plurality of cell culture inserts. As illustrated, a first component 210 defines four receptacles 212a-212d, each configured to receive a cell culture insert. A second component 220 includes a surface 222 configured to define a single elongated channel that, at least partially, defines a flow path that exposes all four inserts to a fluid flow. Alternatively, the second component can include two or more surfaces that define parallel channels, and the first component can include receptacles in a parallel configuration. For example, as shown in FIG. 12, a device 500 includes a first component 510 with receptacles 512a-f in a parallel configuration and a second component 520 with two surfaces 522a-b for defining parallel flow channels beneath the receptacles 512a-f. Returning to FIG. 5, a sealing structure 280 is configured to be disposed between the first and second components to seal a flow path defined by the channel. Similar structures as described with respect to the device 100 can be included in the device 200. While the device 200 is shown with four receptacles, devices can include any number of receptacles (e.g., 2, 3, 4, 5, 10, etc.)

Example embodiments of millifluidic devices were designed and validated using mechanical and simulation software and were fabricated out of acrylic, as further described in Example 1 herein.

FIGS. 6A and 6B illustrate computational modelling of fluid flow through the device 200. As illustrated, a velocity of fluid through the channel 282 remains consistent beneath each of the four receptacles. As is visible in the figures, developing and insteady fluid flow at the inlet is stabilized and becomes stead/uniform prior to the flow reaching the first of the four receptacles.

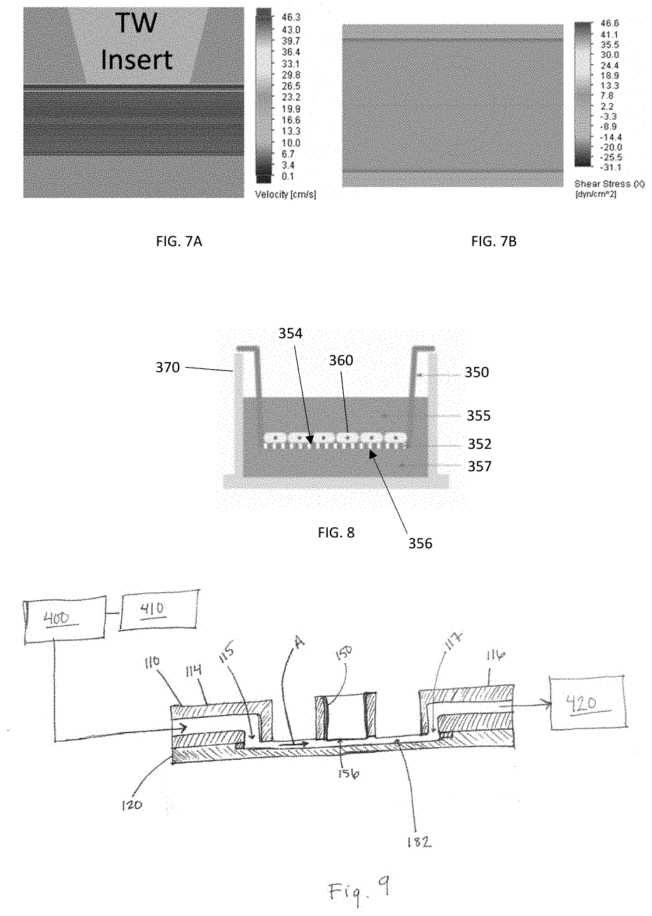

FIGS. 7A and 7B illustrate computational modelling of fluid flow through the device 100 from side (FIG. 7A) and bottom-up (FIG. 7B) views. As illustrated, fully-developed flow patterns and shear stress comparable to those experienced in vivo can be obtained.

In use, cells can first be cultured in a hanging cell culture insert at a user's discretion. For example, endothelial cells can be cultured on an outside/basolateral side of a membrane of the insert. When the cells are ready to be inserted into the device (e.g., when endothelial cells achieve confluency), the cell culture insert can be placed within the receptacle of the device (e.g., receptacle 112). The device can be preassembled or can be assembled upon insertion of the insert. For example, once the cell culture insert is in place within the receptacle, a gasket (e.g., sealing structure 180) can then be placed between the first and second components (e.g., components 110, 120), and the first and second components can be secured in their coupled state (e.g., via screws at 140). Securing the first component to the second component with the inclusion of the gasket creates a fluid-tight flow channel through the device. After the first component is secured to the second component, a lid (e.g., third component 170) with a gasket (e.g., sealing structure 180) underneath can be secured to the first component. The millifluidic device can then be attached to a flow system through inlet and outlet structures. Experiments can successfully last for 24+ hours.

The disclosed devices and systems provide for several advantages and are suitable for use in a variety of applications. The devices are cost-efficient and easy-to-use. The devices can be manufactured with transparent materials to provide for a transparent device and can be used to study complex tissues and organs, such as the brain, heart, and gut in an in vitro setting. The devices advantageously provide for use with commonly employed hanging cell culture inserts (e.g. Transwell® inserts) to create 3-dimensional, multicellular constructs while simultaneously applying controlled levels of fluid flow. Thus, the devices are able to replicate relevant in vivo systems in an in vitro setting, providing a cost-efficient tool with broad customization. Furthermore, with enlarged dimensions compared to common microfluidics, the provided devices can eliminate typical problems associated with microfluidics, including cell seeding and nutrient diffusion challenges. Lastly, use of such devices can be applied to various areas of research, including, but not limited to, basic science, disease mechanism investigation, and drug discovery.

Additional advantages of the described devices include that the devices can be placed on a standard microscope stage for live imaging and real-time studies. The devices can be compatible with molecular assays including fluorescence microscopy, western blotting, ELISAs, and rt-PCR. Proper nutrient diffusion (e.g., oxygen) can be provided. The process of plating cells can be made easier and more consistent because this process can be done outside of the fluidic device, without the need to perfuse cell suspensions.

The provided devices are compatible with complex, multicellular systems. By enabling modelling and analysis of complex cell cultures, such devices can provide for a wide variety of uses. For example, the provided devices can be used to model and study any of the following: 1) the vasculature via smooth muscle cell/pericyte, and endothelial cell co-cultures; 2) the blood-brain barrier via astrocytes, pericytes, and endothelial cells; 3) the gut via epithelial cells and microbiome; 4) the heart via endothelial cells, cardiomyocytes, and fibroblasts; 5) the lung via lung epithelial and endothelial cells; 6) the kidney via kidney epithelial and endothelial cells; 7) reproductive tissues such as the ovaries, cervix, and vas deferens; 8) cancer cell intravasation and extravasation in the vasculature or lymphatic system; 9) therapeutic agents screened against their target tissues/organs.

The devices can accommodate commonly used Transwell inserts, which are relatively inexpensive and have been used for decades to create 3-D cell culture systems, or other hanging cell culture inserts. Therefore, complex cell culture systems that accurately model in vivo systems can be used to increase the translational accuracy of results. The devices can also be reusable and can be compatible with various flow pumps (both centrifugal and peristaltic) and, therefore, a separate flow system is not required, thereby further significantly reducing the cost for users.

An example application of the provided devices is to study the physiology of various tissues and organs, as well as the pathology of diseases associated with those tissues and organs. Additional, secondary applications of the provided devices include drug screening, particularly for drugs targeting the blood-brain barrier, and development of a “body-on-a-chip” device, which can include multiple organ analogs on the same device. More specifically, a blood-brain barrier model using this device can serve as a tool for assessing drugs for the ability to transport across the blood-brain barrier, which is notoriously difficult. Currently, evaluating drugs on their ability to cross the blood-brain barrier is difficult as in vivo models are expensive and complex, while current in vitro models are not able to generate the same permeability regulation that occurs in vivo. In example uses of the disclosed device, the combination of a 3D, multicellular blood-brain barrier construct with physiologically relevant flow patterns provides a blood-brain barrier model that more accurately represents in vivo parameters. Additionally, the devices can provide for modeling of not only individual organ systems, but also modelling of organ systems on a same chip, often referred to as “body-on-a-chip” microfluidics and currently an area of great interest.

The provided devices can be used by individuals with limited cell culture experience, particularly in a fluidic setting. Many common microfluidic devices experience issues with cell seeding due to their small dimensions and need to be seeded using perfusion. However, the larger dimensions and hanging cell culture format of the described devices solve these issues. Similarly, the diffusion of nutrients, including oxygen, is limited in many common microfluidic devices, also due to the small dimensions of these devices. Adequate nutrient diffusion can be more easily achieved with the provided devices over commonly used microfluidic devices. Additionally, while immunofluorescence is the main analytical assay compatible with most microfluidics, the provided devices are compatible with other assays, including, for example, western blotting, ELISAs, and rt-PCR. This significantly broadens the potential applications of the provided devices.

### EXEMPLIFICATION

### Example 1. Blood Brain Barrier (BBB) Model with Prototype Device

1.1 Materials and Methods

1.1.1 Cell Culture

HBMECs were purchased from Cell Systems (Kirkland, Wash.) and used between passages 6 and 8. Cells were cultured in Endothelial Cell Growth Media MV2 from PromoCell (Heidelberg, Germany) supplemented with penicillin-streptomycin (100 U/mL and 100 μg/mL, respectively) and the MV2 SupplementPack, which includes fetal calf serum (0.05 mL/mL), recombinant human epithelial growth factor (5 ng/mL), recombinant human basic fibroblast growth factor (10 ng/mL), insulin-like growth factor (20 ng/mL), recombinant human vascular endothelial growth factor 165 (0.5 ng/mL), ascorbic acid (1 μg/mL), and hydrocortisone (0.2 μg/mL). Primary human brain vascular PCs were purchased from ScienCell (Carlsbad, Calif.) and used between passages 3 and 6. Cells were cultured in ScienCell Pericyte Medium supplemented with fetal bovine serum (0.02 mL/mL), pericyte growth supplement (0.01 mL/mL), and penicillin-streptomycin (100 U/mL and 100 μg/mL, respectively). Primary human ACs isolated from the cerebral cortex were purchased from ScienCell (Carlsbad, Calif.) and used between passages 4 and 7. Cells were cultured in ScienCell Astrocyte Medium supplemented with fetal bovine serum (0.02 mL/mL), astrocyte growth supplement (0.01 mL/mL), and penicillin-streptomycin (100 U/mL and 100 μg/mL, respectively). Cell culture flasks for PCs and ACs were coated with poly-L-lysine (2 μg/cm² in water) for at least 1 hour and up to 24 hours prior to being plated with PCs and ACs. In addition to HBMEC, PC, and AC cell cultures, human aortic endothelial cells (HAECs) acquired from PromoCell (Heidelberg, Germany) were also utilized to validate flow patterns generated by the millifluidic device. HAECs were utilized for millifluidic validation instead of HBMECs as the impact of flow on HAECs has been robustly investigated in the past while less research has been performed using HBMECs. Particularly, HAECs are well known to align in the direction of fluid flow exposure while the response of HBMECs to fluid flow is less clear. HAECs were similarly cultured in PromoCell Endothelial Cell Growth Media MV2 as previously described. All cell types were cultured in a humidified incubator maintained at 37° C. and 5% CO₂.

1.1.2 Transwell Blood-Brain Barrier Model Development

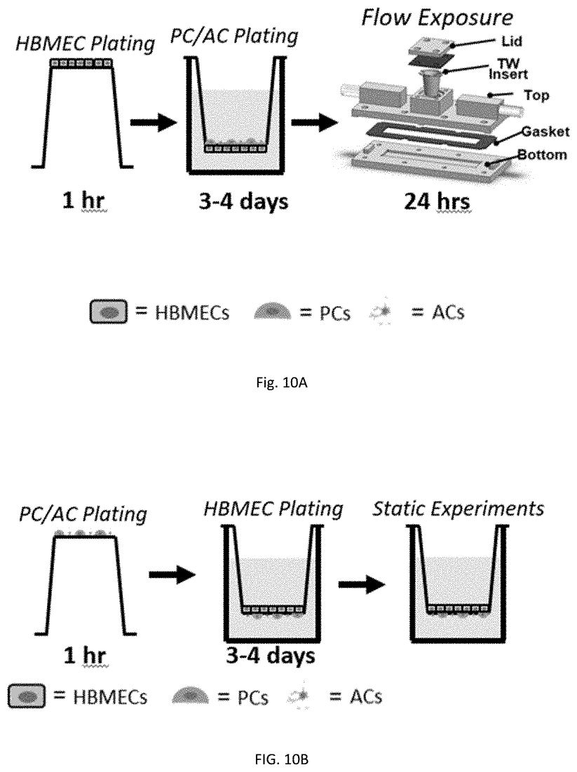

A BBB model containing HBMECs, PCs, and ACs was developed using 24-well Transwell inserts (FIGS. 10A-10B). To develop the model, 5×10⁵ HBMECs were first plated on the 0.33 cm² area of the abluminal side of inverted, fibronectin-coated (15 μg/cm²) Transwell insert membranes (HBMEC plating density of 1.5×10⁶ cells/cm²). The inverted Transwell inserts were then placed into a humidified incubator at 37° C. and 5% CO₂ for one hour to allow for cell attachment. Following the one-hour incubation period, the Transwell inserts were then inverted to their right-side-up positions and placed into the wells of 24-well plates containing 700 μL of PromoCell MV2 media. At this time, 5×10⁵ PCs and 5×10⁵ ACs were plated onto the luminal side of Transwell insert membranes in the absence of a fibronectin coating and covered with 300 μL of ScienCell media (150 μL of each PC and AC media). Cells were then placed into the incubator and allowed to grow for 3-4 days with media changes taking place every other day. The chosen seeding densities were utilized to obtain an approximate 1:1:1 ratio of HBMECs:PCs:ACs upon cell confluency. In addition to the HBMEC/PC/AC co-culture BBB model, HBMEC monoculture, HBMEC/PC co-culture, and HBMEC/AC co-culture models were also developed to determine both the impact of PCs versus ACs on HBMEC phenotype and the impact of shear stress on HBMEC monolayers. These models were developed as described above but with either no cells on the luminal membrane or only one cell type (either PCs or ACs) on the luminal membrane. It should also be noted that for static dextran permeability assays the BBB cell organization was revised from what is described above, and revisions are described below where appropriate.

1.1.3 Design and Fabrication of Blood-Brain Barrier Millifluidic System

ECs of the previously described BBB model containing HBMECs, PCs, and ACs or HBMEC monolayers were exposed to a continuous, physiologically relevant shear stress of 12 dynes/cm² for 24 hours using a custom, Transwell-compatible, millifluidic device as detailed below. While previous BBB microfluidics, which are commonly made out of PDMS, are often limited by issues of nutrient diffusion and air bubble formation; the use of immortalized cell lines or non-human primary cells; and a lack of adaptability for downstream analytical techniques such as high magnification fluorescence microscopy, this device overcomes these limitations due to its compatibility with Transwell inserts, which have been utilized for decades to study BBB function. The millifluidic device was fabricated out of acrylic and designed using SolidWorks (in accordance with the schematic shown in FIG. 1). The flow channel within the device measures 70×13×0.5 mm (L×W×H). To assemble the device, a Transwell insert is placed into the top of the millifluidic device, which contains precisely designed notches to allow for the Transwell membrane to align flush with the flow channel. The chamber top is then attached to the chamber bottom, with a silicone gasket in between to prevent leakage, using screws. A lid and additional silicone gasket are then secured directly above the Transwell insert to prevent leakage from this orifice. The system is then connected to a peristaltic pump. A media reservoir, for CO₂ diffusion, and a pulse dampener, to reduce flow pulsatility, were included in the flow loop. The flow patterns and shear stress magnitudes that were generated were validated via SolidWorks Flow Simulation. During shear stress exposure, the flow system, with the exception of the peristaltic pump, was contained within a humidified incubator at 37° C. and 5% CO₂.

1.1.4 Live Fluorescent Particle Tracking for Millifluidic Validation

To confirm flow patterns within the millifluidic device, live tracking of fluorescent particles was performed. Specifically, fluorescent polystyrene microspheres (Bangs Laboratories; Fishers, Ind.) with an average diameter of 1 μm were diluted in water at a 1:1000 dilution, which was then perfused through the millifluidic device. Particle movement was subsequently tracked via the use of a Zeiss Axio Observer Z1 fluorescent microscope.

1.15 HBMEC Flow Exposure Using a Previously Fabricated Parallel Plate Flow Chamber

In addition to the Transwell-compatible millifluidic device, a previously fabricated parallel plate flow chamber was also utilized, particularly to investigate the impact of flow on HBMEC alignment. This device is based on a modified parallel plate flow chamber adapted from previous work, described in I. C. Harding, R. Mitra, S. A. Mensah, I. M. Herman and E. E. Ebong, J Transl Med, 2018, 16. For these experiments, HBMECs were plated on fibronectin-coated (15 μg/cm²) glass coverslips and incorporated into the flow chamber for 24 hours of shear stress exposure at 12 dynes/cm².

1.1.6 Dextran Permeability Assay for Cell Culture Studies

To assess the mono- and co-culture systems for barrier function, a dextran permeability assay using Texas Red-conjugated 40 kDa dextran and 3 kDa dextran (Thermo Fisher; Waltham, Mass.) was performed. Assays were performed on cell culture models after 3-4 days of culture in static conditions. For samples pre-conditioned with flow, the organization of HBMECs, PCs, and ACs is as described above (FIG. 10A). Alternatively, for non-pre-conditioned samples, the organization of HBMECs, PCs, and ACs was reversed: PCs and ACs were plated on the abluminal Transwell membrane surface while HBMECs were plated on the luminal surface (FIG. 10B). For both static and flow-conditioned samples, 40 kDa dextran was added to the luminal Transwell compartment at a concentration of 0.5 mg/mL in 200 μL of PromoCell MV2 medium. The same procedure was followed for the 3 kDa dextran except 0.25 mg/mL was added to the luminal compartment to conserve dextran. The abluminal compartment was filled with 700 μL of the appropriate medium, depending on the model used (mono- or co-culture). Sixty minutes after the dextran was introduced to the system, 50 μL of media from the abluminal compartment was collected. The collected media was analyzed for fluorescent dextran content using a Molecular Devices SpectraMax i3× plate reader. Dextran concentration within experimental samples was determined by comparison to fluorescent values obtained from a standard curve. The apparent permeability was then calculated using Equation 1:

$${P}_{app} = \frac{{C}_{a} * {V}_{a}}{t * s * {C}_{l}} \qquad ( 1 )$$

where Pₐₚₚ is the apparent permeability, Cₐ is the measured abluminal dextran concentration, Va is the abluminal media volume, t is time, s is surface area and C₁ is the initial luminal dextran concentration. To determine the permeability coefficient of the BBB model independent of the porous Transwell membrane, the permeability coefficient of a blank, fibronectin-coated Transwell membrane was determined. The permeability coefficient of the BBB model was then calculated using Equation 2:

$$\frac{1}{{P}_{app}} = \frac{1}{{P}_{M}} + \frac{1}{{P}_{BBB}} \qquad ( 2 )$$

where P_{M} is the permeability coefficient of the cell-free Transwell membrane and PBBB is the permeability coefficient of the BBB model.

1.1.7 Transepithelial Electrical Resistance for Cell Culture Studies

Transepithelial electrical resistance (TEER) of HBMECs monocultures was measured to assess barrier function. Before testing, proper media volumes in both the luminal (300 μL) and abluminal (700 μL) compartments were ensured. TEER was measured using a World Precision Instruments Epithelial Volt/Ohm Meter (EVOM2) and chopstick electrode. Three measurements for each sample were collected to provide increased accuracy. The average of the three measurements was used for statistical analysis.

1.1.8 Immunocytochemistry

To ensure proper cell growth and phenotype within the BBB model, including EC monolayer formation and the presence of astrocyte foot processes, the BBB model was evaluated using immunocytochemistry via the following cell specific markers: platelet-EC adhesion molecule-1 (PECAM-1), neural/glial antigen 2 (NG2), and glial fibrillary acidic protein (GFAP) for HBMECs, PCs, and ACs, respectively. Additionally, HBMECs were also analyzed for junctional integrity via immunocytochemistry of ZO-1 and claudin-5. Prior to immunocytochemistry, cells labeled for PECAM-1, NG2, or GFAP were fixed in 4% paraformaldehyde for 20 minutes at room temperature. Subsequently, samples were first permeabilized with 0.5% Triton X-100 for 5 minutes at room temperature and then blocked with 5% goat serum for 1 hour at room temperature. Alternatively, cells labeled for ZO-1 and claudin-5 were fixed in ice-cold methanol for 5 minutes at −20° C. and then blocked in 5% goat serum for 1 hour at room temperature. All samples were then incubated with the following primary antibodies diluted in blocking solution overnight at 4° C.: rabbit anti-PECAM-1 (1:400; Novus Biologicals; Centennial, Colo.), mouse anti-NG2 (1:100; eBioscience; San Diego, Calif.), rat anti-GFAP (1:1,500; Invitrogen; Waltham, Mass.), rabbit anti-ZO-1 (1:200; Invitrogen; Waltham, Mass.), and mouse anti-claudin-5 (1:100; Invitrogen; Waltham, Mass.). Samples were incubated with the following secondary antibodies diluted in phosphate buffered saline for 1 hour at room temperature: goat anti-rabbit Alexa Fluor 647 (1:500; Invitrogen; Waltham, Mass.), goat anti-mouse Alexa Fluor 488 (1:500; Invitrogen; Waltham, Mass.), and goat anti-rat Alexa Fluor 546 (1:1,500; Invitrogen; Waltham, Mass.). To label cell nuclei, samples were incubated with 4′, 6-Diamidine-2′-phenylindole (DAPI) dihydrochloride at a concentration of 300 nM for 5 minutes at room temperature. Inserts were placed on glass bottom petri dishes (Cellvis; Mountain View, Calif.) with a #0 coverslip thickness. Samples were then imaged using a Zeiss LSM 880 confocal microscope with 20× and 40× (water immersion lens) magnification objectives.

1.1.9 Western Blotting

For western blot analysis, HBMECs cultured on the abluminal side of Transwell membranes were lysed using radioimmunoprecipitation assay buffer containing 150 mM sodium chloride (NaCl), 1% Triton X-100, 50 mM Tris base, 0.1% sodium dodecyl sulfate (SDS), 5 mM ethylenediaminetetraacetic acid, 1 mM phenylmethylsuphonyl fluoride, and Roche cOmplete EDTA-free protease inhibitor cocktail. Prior to SDS polyacrylamide gel electrophoresis (SDS-PAGE), HBMEC protein lysates were prepared in Lamelli buffer containing 50 mM dithiothreitol and boiled at 95° C. for 5 minutes. Protein lysates were then run on 7.5% SDS-PAGE gels and wet transferred to polyvinylidene difluoride (PVDF) membranes. It should be noted that the concentration of protein obtained from 0.33 cm² surface of the Transwell inserts was low due to limited cell content. Therefore, to accommodate the subsequently low quantity of protein loaded on SDS-PAGE gels (˜5 μg per well), a Pierce Western Blot Signal Enhancer was utilized following the manufacturer's protocol to amplify the signal of all proteins probed on the PVDF membranes except β-actin protein, which was utilized as a housekeeping protein. Membranes were blocked using 5% milk solution and probed with primary antibodies overnight at 4° C. on a rocker at the following dilutions: ZO-1 (1:750; Invitrogen; Waltham, Mass.), occludin (1:1000; Invitrogen; Waltham, Mass.), claudin-5 (1:1000; Invitrogen; Waltham, Mass.), VE-cadherin (1:1000; BioLegend, San Diego, Calif.), caveolin-1 (1:1000; Santa Cruz Biotechonology; Dallas, Tex.), and B-actin (1:3,000; Invitrogen; Waltham, Mass.). These proteins were probed due to their implication in BBB permeability. All samples were incubated with species-appropriate, HRP-conjugated secondary antibodies for 1 hour at room temperature on a rocker at a 1:3,000 dilution in blocking buffer, except for β-actin, which utilized a 1:10,000 secondary antibody concentration. For chemiluminescent detection, samples were incubated in BioRad Clarity ECL reagents for 5 minutes at room temperature and imaged using a BioRad ChemiDoc Touch Imaging System.

1.1.10 Statistical Analysis

All data is presented as mean±standard error of the mean. Prior to statistical analysis, western blot data was normalized to the housekeeping gene to account for loading differences and subsequently normalized to the control groups within each experiment to eliminate any confounding inter-experiment variables such as cell passage number. Normal distributions of data were confirmed using the Shapiro Wilk test. Subsequently, one-way ANOVAs with post-hoc Tukey's multiple comparison tests were used to identify statistically significant differences between groups. Alternatively, data from dextran permeability assays, specifically for flow-treated samples, was analyzed using a paired t-test to similarly remove bias introduced from inter-experiment variables.

1.2 Results

1.2.1 Validation of the Millifluidic System

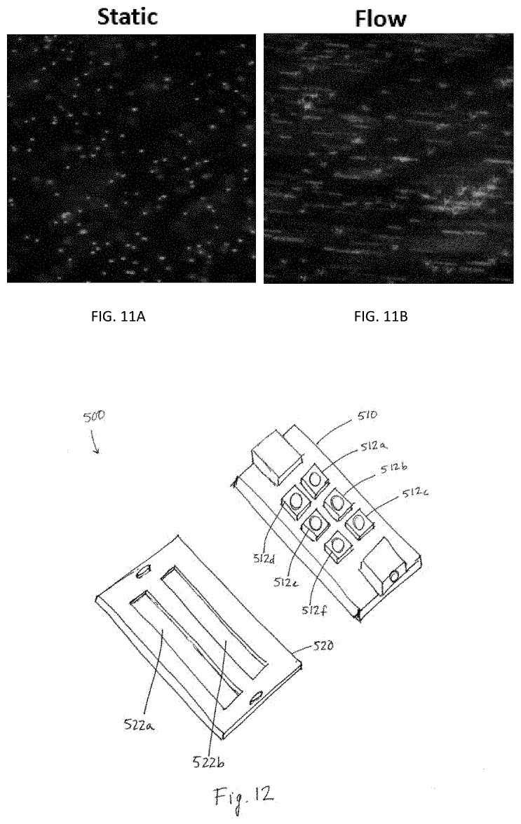

A millifluidic device compatible with commonly used 24-well Transwell inserts was designed to investigate the impact of shear stress exposure on BBB integrity (FIG. 1). The millifluidic device follows the design of a standard parallel-plate flow chamber with a flow channel measuring 70×13×0.5 mm (L×W×H). Flow patterns and associated shear stresses were predicted via SolidWorks Flow Simulation. Specifically, a flow rate of 72 mL/min generated a physiologically relevant shear stress of 12 dynes/cm². Live tracking of fluorescent microbeads was used as a first validation step, to ensure that flow patterns predicted by the computational model translated into practice (FIGS. 11A-B). As a second validation step, the system was scanned for the presence of microbubbles. No microbubble formation was observed during testing or use of the device.

As a third step in the validation of the Millifluidic System, we sought to validate the BBB millifluidic system by using it to investigate the impact of shear stress application on HBMEC alignment, a common EC mechanobiological response. However, several previous studies did not observe brain EC alignment in the direction of flow. Therefore, to confirm proper flow patterns and resulting shear stresses within the millifluidic device using cell alignment, the device was tested using HAECs, which are well-characterized and known to align in the direction of flow exposure. Consistent with the literature, we found that HAECs did indeed align in the direction of flow after 24 hours of shear stress exposure at 12 dynes/cm². These results validate the flow patterns and shear stresses predicted by the computational model.

In parallel studies we applied the same shear stress to HBMEC monolayers to determine if they could align at all, despite the previous reports of absence of brain EC alignment in the direction of flow. We used a previously fabricated, well-characterized, parallel-plate flow chamber in which the HBMECs were cultured on fibronectin-coated glass coverslips at low versus high cell densities before exposure to uniform flow. This model was used for simplicity, and we acknowledge that the substrate on which cells are grown (glass coverslip vs. polycarbonate membrane) may also influence cells' alignment. We found that resultant cell alignment parallel to the direction of flow did occur for HBMECs but depended on cell density at the time of flow introduction. Cells exposed to flow at a lower cell density (˜562 cells/mm²) aligned in the direction of flow statistically significantly when compared to static controls and consistent with previous studies on ECs from different vascular beds. An average of 22.3±1.0% of cells aligned within 15° of the axis parallel to flow while 8.1±0.6% of cells aligned within 15° of the axis perpendicular to flow. Cells exposed to flow at a higher cell density (1174 cells/mm²) exhibited a decreased tendency to align with flow, which was statistically different from both static and low cell density uniform flow samples. In high cell density samples, only 18.4±3.3% of cells aligned within 15° of the flow axis while 11.8±2.2% of cells aligned within 15° of the axis perpendicular to flow.

The dependency of HBMEC alignment on cell density may be explained by the fact that EC migration rate is reduced at higher cell densities. Thus, cells at higher densities may require additional flow exposure time to adjust their configuration and achieve the same level of cell alignment. In addition, the fact that alignment of HBMECs depends on cell density while cell density is not a concern for successful HAEC alignment suggests that differences in alignment propensity exist between ECs of different vascular beds. However, the demonstration that HBMECs can indeed align in the direction of flow after 24 hours of exposure is new and valuable information given that several previous studies did not observe brain EC alignment in the direction of flow. Future validation of the BBB millifluidic system by investigating EC alignment in response to shear stress stimulus should utilize HBMEC and not simply HAECs or other ECs that have been historically well known for alignment.

1.2.2 Validation of BBB Model Embedded in the Millifluidic System

After validating proper function of the millifluidic system via live tracking of fluorescent microbeads, by scanning for microbubbles, and by investigating cell alignment, attention was turned to the BBB model embedded within it. First, to determine the ideal culture time of BBB constructs, TEER of HBMEC monolayers was measured. We found that HBMEC TEER increased ˜80 Ohms*cm² to ˜97 Ohms*cm² over a three-day period but subsequently remained stable. Thus, all BBB models, which contained primary HBMECs, human PCs, and human ACs at an approximate 1:1:1 HBMEC:PC:AC ratio, were cultured for 3 or 4 days before experimentation.

Secondly, visual validation of the BBB model was performed. This was made possible via high magnification (20×, 40×) imaging of immunofluorescent cell-specific markers, specifically PECAM-1, NG2, and GFAP targeting HBMECs, PCs, and ACs, respectively. Monolayers of ECs formed on the abluminal membrane of the Transwell inserts as determined by confocal microscopy. Integrity of the EC monolayer was confirmed by strong PECAM-1 signal localized to cell-cell junctions. On the luminal membrane, NG2 and GFAP fluorescence confirmed the presence of PCs and ACs while highlighting astrocyte foot processes extending from the AC cell body throughout the co-culture model. 3D projections of the co-culture convey the multicellular geometry of the model.

1.2.3 Application of Millifluidic System to Show that, Under Static Conditions, Addition of Astrocytes and Pericytes to Endothelial Cell Monolayers Strengthens BBB Model Barrier Integrity

The contributions of PCs and ACs to barrier integrity of the BBB model were investigated via a dextran permeability assay. Normalizing the permeability coefficients to the average of the EC monolayer samples, for every experiment, negated the effects of cell passage number and membrane material. For the 40 kDa dextran permeability analysis, the individual addition of PCs to HBMECs as well as ACs to HBMECs decreased the permeability of the BBB model in comparison with the HBMEC monolayer. Specifically, EC/PC samples had a normalized mean permeability coefficient of 0.320±0.0441, a statistically significant 3-fold decrease from HBMEC monolayers. A similar 3-fold decrease from HBMEC monolayers was observed in EC/AC samples, which had a normalized mean permeability coefficient of 0.328±0.0538. We also found that EC/PC/AC co-cultures had a normalized permeability coefficient of 0.330±0.100, which was significantly lower (decreased permeability) than what was found for HBMEC monolayers but not statistically different from what was found for EC/AC or EC/PC co-cultures. To further investigate barrier integrity of the various cultures, 3 kDa dextran permeability assays were also performed. Like the 40 kDa dextran, permeability was reduced in the EC/PC, EC/AC and EC/PC/AC co-cultures with normalized permeability values of 0.363±0.0608, 0.683±0.0519, and 0.521±0.0337 respectively. Interestingly, EC/PC samples exhibited significantly lower permeability than the EC/AC and EC/PC/AC samples while EC/AC samples had a significantly higher permeability than the other two samples). This data from the permeability assays indicates that both PCs and ACs help improve BBB integrity.

Over recent decades, many studies have identified beneficial roles of both PCs and ACs in BBB regulation. For example, the addition of PCs and ACs to brain EC monolayers has been shown to reduce permeability to fluorescent dextran. In agreement with these findings, we observed a statistically significant reduction in dextran permeability in the EC/AC, EC/PC, and EC/PC/AC co-cultures compared to HBMEC monolayers, suggesting a beneficial role of both ACs and PCs. However, we did not observe a compounding effect on permeability when both ACs and PCs were cultured with HBMECs using the 40 kDa dextran. This may be due to the fact that the individual addition of either ACs or PCs leads to strong barrier formation in which permeability to relatively large molecules (e.g., 40 kDa dextran) has already been sufficiently impeded.

To further investigate this threshold phenomena, a smaller 3 kDa dextran molecule was utilized in a subsequent permeability assay. Contradictory to the 40 kDa experiment, a significant decrease in permeability was observed in the EC/PC condition compared to the EC/AC and EC/AC/PC. Furthermore, EC/AC cultures demonstrated a significantly higher permeability compared with the other two. This result indicated that pericytes may be playing a stronger role in barrier integrity compared to astrocytes although they both decrease permeability compared to the HBMEC monolayer. For the pericyte co-culture, this observation agrees with previous findings where it was found that pericytes are critical in promoting TJ protein expression while reducing endothelial transcytosis through the inhibition of molecules which increase vascular permeability. Astrocytes on the other hand have a more complex relationship with BBB permeability and may play a dual-role in overall barrier integrity. Astrocytes have been shown to release a variety of factors which ultimately affect the expression of TJ proteins like ZO-1, claudin-5, and occludin. Astrocyte-derived factors which may increase vascular permeability include VEGF, MMPs, and NO. Conversely, several astrocyte-derived factors have demonstrated protective barrier properties like ANG-1 and SHH. These opposing molecular pathways may be the reason why the EC/AC co-culture exhibits greater permeability than the EC/PC condition. It is well-established that smaller dextran molecules will lead to greater permeability in the BBB. For this reason, small differences in barrier integrity may become more apparent when a smaller molecule permeates through the BBB model. The relative difference in permeability observed in the co-cultures in the 3 kDa assay compared to the 40 kDa assay may be a result of a barrier threshold being reached for the larger molecule. Regardless, both astrocytes and pericytes decrease permeability compared to the EC monolayer.

1.2.4 Flow Stimulates Improved Barrier Integrity of the HBMEC Monolayer but has Negligible Effects on Integrity of the HBMEC/PC/AC co-culture BBB Model

We also investigated the impact of flow exposure on BBB barrier integrity. Interestingly, the application of flow for 24 hours at a shear stress of 12 dynes/cm² to the EC/PC/AC co-culture BBB model had no impact on dextran permeability. When compared to static controls, BBB flow exposure only reduced permeability by 3.83% from 1.88×10⁻⁷±3.53×10⁻⁸ cm/s to 1.81×10⁻⁷±4.01×10⁻⁸ cm/s, which was statistically insignificant. However, because ECs have been shown to benefit from shear stress exposure, the same experiment was performed on HBMEC monolayers. In this case, shear stress exposure successfully reduced permeability to 40 kDa dextran by a statistically significant 21.0% compared to static controls from 4.06×10⁻⁷±7.62×10⁻⁸ cm/s to 3.21×10⁻⁷±6.94×10⁻⁸ cm/s.

Our flow exposure studies demonstrate that while shear stress reduces permeability in HBMEC monolayers, it has no impact on the EC/PC/AC co-culture BBB model. We postulate that the observed findings in the EC/PC/AC co-culture BBB model may be due to insufficient EC expression of mechanotransducers, which sense and respond to mechanical stimuli. For example, insufficient endothelial glycocalyx, a known mechanotransducer, has been shown to regulate EC permeability. Thus, enhanced EC glycocalyx expression in the BBB co-culture model may be required to enable the ECs' ability to sense and respond to fluid flow and thus their ability to down regulate permeability. Alternatively, the observed results could be due to changes in EC/PC/AC communication when comparing static to flow conditions. For example, the astrocyte-derived factors which may increase permeability like VEGF, MMPs and NO may be upregulated in flow conditions. An upregulation of these factors could mitigate the positive effects of flow seen with the EC monolayer alone. Finally, the reported discrepancy on the effects of shear stress on barrier integrity between HBMEC monolayers and the BBB co-culture could be due to size of the tracer molecule used. Permeability data for the 3 kDa tracer molecule was also collected comparing monolayer and EC/PC/AC cultures in static and flow conditions. These results did not show a statistical difference between static and flow conditions for either culture. Regardless of the mechanism, this discrepancy should be further investigated in the future. The device that has been described herein will enable such future studies to further our understanding of BBB regulation in both physiological and pathological conditions.

1.2.5 Pericytes Reduce HBMEC Occludin Expression while Astrocytes have No Significant Impact on HBMEC Expression of Permeability Regulating Proteins

To identify potential molecular mechanisms responsible for the observed changes in BBB permeability as a result of PC/AC co-culture or flow exposure, protein level analysis via western blotting was performed. In static samples, western blotting identified a statistically significant decrease in occludin expression in HBMECs from EC/PC co-cultures (41.2±3.5% decrease) and EC/PC/AC co-cultures (43.2±7.3% decrease) as compared to HBMEC monolayers. In EC/AC co-cultures, a 14.4±9.6% reduction in occludin expression was also observed, but this was not statistically significant. Collectively, this data suggests that the addition of PCs to HBMEC monolayers may actually decrease HBMEC occludin expression, while the impact of ACs is unclear. In addition to the observed decrease in occludin expression, we also most notably identified changes in claudin-5 expression. Specifically, we identified increased claudin-5 expression in EC/PC, EC/AC, and EC/PC/AC conditions of 53.6±24.6%, 35.3±27.1%, and 42.2±43.1%, respectively. While these changes were not statistically significant, the trends suggest that both PCs and ACs may increase the expression of claudin-5 upon co-culture. With regard to ZO-1 expression, we additionally observed a 25±16.8% increase in HBMECs from EC/PC co-cultures when compared to HBMEC monolayers. However, this increase was not statistically significant. The expression of VE-cadherin and caveolin-1 was also analyzed, but no significant changes in either protein were observed.

The impact of flow exposure on HBMEC tight junction protein expression was also investigated. The impact of flow on VE-cadherin and caveolin-1 expression was not investigated as no substantial changes in the expression of these proteins were observed in the static mono- and co-culture BBB models. In EC/PC/AC co-cultures, we found that flow exposure led to a statistically significant increase in HBMEC ZO-1 expression (22.2±5.8% increase) compared to static conditions. In contrast, flow exposure in HBMEC monolayers led to a negligible 1.9±13.6% increase in ZO-1 expression, which was not statistically significant. Additionally, in both the co-culture and monolayer models, shear stress application resulted in a statistically significant reduction in both claudin-5 and occludin expression. Particularly, occludin expression was reduced by 44.1±5.2% and 54.2±6.3% in EC/PC/AC co-cultures and HBMEC monolayers, respectively, while claudin-5 expression was reduced by 24.7±4.4% and 45.9±9.3%, respectively.

Previous studies implicating PCs and ACs in regulating BBB integrity, specifically BBB permeability, have typically attributed reduced permeability to increased expression of tight junction proteins such as ZO-1, claudin-5, and occludin. The contribution of other proteins to barrier integrity, such as VE-cadherin and caveolin-1, have also been investigated to lesser extents. Here, we found that the addition of both PCs and ACs to HBMECs led to the increased expression of claudin-5, albeit statistically insignificant. We also observed increased ZO-1 expression in EC/PC co-cultures when compared to HBMEC monolayers. These results collectively suggest that the decreased dextran permeability following the addition of PCs or ACs to HBMECs may be the result of increased claudin-5 and/or ZO-1 expression. However, we also observed a statistically significant decrease in occludin expression in both EC/PC and EC/PC/AC conditions, suggesting that PCs may interestingly reduce occludin expression despite simultaneously reducing BBB permeability. These results highlight the complex regulation of BBB permeability, which depends on the expression and function of dozens of proteins. Thus, PCs and ACs may also regulate BBB permeability through alternative proteins not investigated in this study. These may include ABC transporters, such as P-glycoprotein, integrins, or junctional adhesion molecule, such as JAM-A, all of which have been implicated in regulating BBB permeability. We similarly hypothesize that the reduced dextran permeability following flow exposure of HBMEC monolayers may be due to these alternative mechanisms as we did not observe any significant increases in junctional protein expression following flow exposure. Future studies should investigate these and other mechanisms of BBB regulation by flow.

1.2.6 Static and Flow-Exposed HBMEC Monolayers and BBB Co-Cultures are Characterized by Strong Junctional Expression of ZO-1 and Claudin-5

Immunocytochemistry was performed in both static and flow-exposed HBMEC monolayers and BBB co-cultures to identify the junctional localization of ZO-1 and claudin-5. ZO-1/claudin-5 co-staining in HBMEC monolayers as well as EC/PC, EC/AC, and EC/PC/AC co-cultures demonstrated substantial junctional localization of both proteins as anticipated. While HBMEC monolayers, EC/PC co-cultures, and EC/PC/AC co-cultures demonstrate strong claudin-5 staining, claudin-5 expression in EC/AC co-cultures seemed diminished. However, expression and junctional localization of ZO-1 in these samples remained strong. Consistent with western blotting data, co-staining of flow-exposed HBMEC monolayers and EC/PC/AC co-cultures demonstrated a significant reduction in claudin-5 expression. An increase in ZO-1 expression in flow-exposed EC/PC/AC co-cultures can also be observed. Interestingly, shear stress application to the co-culture BBB model, and perhaps to the HBMEC monolayer to a lesser extent, appears to increase the junctional thickness of ZO-1 expression. This observation highlights the importance of a high expression level for this protein and not solely its distribution and localization at junctions.

1.3 Summary of Results

This study describes a novel millifluidic device that is both easy to utilize and compatible with numerous upstream and downstream experimental tasks, as summarized below.

First, cell seeding and culturing can be problematic in common (e.g. PDMS) microfluidic devices. In contrast, the compatibility of our device with Transwell inserts allows for easy cell seeding and culturing including co-culturing of multiple cell types. Second, common microfluidics fabricated out of PDMS are often limited by issues with microbubble formation. The fabricated millifluidic device avoids microbubble formation via the use of larger channel dimensions. Third, common microfluidic devices can also have limited compatibility with downstream analytical techniques such as high magnification microscopy and western blotting, but the fabricated millifluidic device circumvents these issues using a design compatible with disassembly. Therefore, as our millifluidic device requires minimal knowledge about the design and is easy to use compared to a microfluidic system, it is a more feasible option for individuals without microfluidic expertise who are interested in investigating shear stress effects on the BBB. Additionally, in contrast with many previous BBB models that utilized non-human or immortalized cell lines and therefore lack physiological relevance, our model contains only primary human cells, which provides increased confidence of results and conclusions that are relevant to human physiology and disease.

We were able to confirm that the millifluidic device induces EC alignment. In addition to the observed impacts of flow on cell alignment, we also found that flow exposure reduced 40 kDa dextran permeability in HBMEC monolayers. Furthermore, using a hanging cell culture BBB model (consisting of HBMECs, human ACs, and human PCs) embedded in the novel millifluidic device, a beneficial role of both ACs and PCs on BBB integrity was identified. These results can be further examined in the future, particularly to investigate the (1) mechanotransducers responsible for the observed impact of flow exposure on BBB integrity and (2) the specific mechanisms through which astrocytes and/or pericytes improve BBB barrier integrity. Collectively, such studies may identify unique therapeutic targets for restoring BBB function in numerous neurological pathologies.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A device for cell culture analysis, comprising:
a first component comprising a receptacle configured to receive a cell culture insert having an apical surface and a basal surface;
a second component;
an inlet port disposed at at least one of the first and second components; and
an outlet port disposed at at least one of the first and second components, the first component and second component being releasably couplable and configured to define a flow path from the inlet port to the outlet port when in a coupled state, the flow path at least partially defined by a surface of the second component, the first component configured to expose the basal surface of the cell culture insert to the flow path.

2. The device of claim 1, further comprising a third component configured to engage with the first component to enclose the cell culture insert in the receptacle.

3. The device of claim 1, wherein the surface of the second component and a complementary surface of the first component define a channel that at least partially defines the flow path.

4. The device of claim 3, wherein one of the first and second components comprises at least one channel support member configured to engage a complementary structure at the other of the first and second components to maintain a height of the channel when pressure is applied to the device.

5. The device of claim 4, wherein the at least one channel support member comprises at least two channel support members, the at least two channel support members disposed at opposing ends of the channel.

6. The device of claim 5, wherein one of the at least two channel support members is disposed adjacent to the inlet port and the other of the at least two channel support members is disposed adjacent to the outlet port.

7. The device of claim 3, wherein the channel is a millifluidic channel.

8. The device of claim 1, wherein the receptacle comprises an alignment structure configured to maintain a position of the basal surface of the cell culture insert with respect to the flow path.

9. The device of claim 1, wherein one of the first and second components comprises at least one alignment member configured to engage a complementary structure at the other of the first and second components to align the first and second components for coupling.

10. The device of claim 9, wherein the at least one alignment member is further configured to be a channel support member.

11. The device of claim 1 wherein the first and second components comprise a transparent material.

12. The device of claim 1, wherein first and second components are reusable, sterilizable, autoclavable, or a combination thereof.

13. The device of claim 1, wherein the device further comprises at least one sealing member disposed between the first and second components and configured to maintain a pressure of the flow channel.

14. The device of claim 1, wherein the cell culture insert is a hanging cell culture insert.

15. The device of claim 1, wherein the first component comprises a plurality of receptacles, each receptacle configured to receive a cell culture insert.

16. The device of claim 15, wherein the surface of the second component and a complementary surface of the first component define at least two channels that at least partially define a flow path.

17. A system for cell culture analysis, comprising:
a device comprising:
a first component comprising a receptacle configured to receive a cell culture insert having an apical surface and a basal surface,
a second component,
an inlet port disposed at at least one of the first and second components, and
an outlet port disposed at at least one of the first and second components, the first component and second component being releasably couplable and configured to define a flow path from the inlet port to the outlet port when in a coupled state, the flow path at least partially defined by a surface of the second component, the first component configured to expose the basal surface of the cell culture insert to the flow path; and
at least one pump in fluidic communication with the inlet port.

18. The system of claim 17, wherein the pump is configured to supply a fluid flow to the fluid path at a flow rate that induces shear stress of cells disposed at the basal surface.

19. The system of claim 18, wherein the flow rate is about 0.1 ml/min to about 120 ml/min.
